(19) United States
(12) Patent Application Publication
(10) Pub. No.: US 2025/0171424 A1
(43) Pub. Date: May 29, 2025

(54) Acid-Base Controlled Nano-Bending Lassos to Manipulate Photo-Switching Units in [1]Rotaxanes

(71) Applicant: National Yang Ming Chiao Tung University, Hsinchu (TW)

(72) Inventors: Hong-Cheu Lin, HSINCHU (TW); Manh-Khang Trang, HSINCHU (TW); Yaw-Kuen Li, HSINCHU (TW)

(21) Appl. No.: 18/935,452

(22) Filed: Nov. 2, 2024

(30) Foreign Application Priority Data
Nov. 27, 2023 (TW) 112145848

(51) Int. Cl. C07D 409/14; A61K 9/107; A61K 41/00

(52) U.S. Cl. CPC C07D 409/14; A61K 9/1075; A61K 41/0057

(57) ABSTRACT

Provided is acid-base controlled nano-bending lassos to manipulate photo-switching units in [1]rotaxanes, which contain sequentially bonded a macrocyclic moiety including a cyclic polyether moiety, a photochromic photo-switching moiety, a first station moiety including a secondary ammonium group, a linear moiety, a second station moiety being capable of forming multiple hydrogen bonding with the macrocyclic moiety, and an aggregation-induced emission stopper moiety. Under the base condition (in the loosened lasso form), the photochromic photo-switching moiety can perform a ring-closing reaction upon ultraviolet light exposure, and a ring-opening reaction can occur when exposed to visible light. However, the mentioned photo-switchable ring-closing and ring-opening reactions cannot proceed under the acidic condition (in the tightened lasso form). A linear moiety is bonded to the first station moiety and passes through the macrocyclic moiety. The photo-switching moiety and the stopper moiety are located on both sides of the macrocyclic moiety.

### CROSS-REFERENCE TO RELATED APPLICATION

This application claims the priority benefit of Taiwan application serial no. 112145848, filed Nov. 27, 2023, the full disclosure of which is incorporated herein by reference.

### BACKGROUND

### Technical Field

The disclosure relates to a molecular switch and its application, in particular to a kind of mechanically interlocked molecules (MIMs) with dual molecular switches.

### Description of Related Art

In past research, attempts were made to use ultraviolet light and visible light to carry out photochromic reversible responses of diarylethene (DAE) structure, and use non-covalent bonding interactions, such as host-guest supramolecular and multiple hydrogen bonds (or stacking of π-π interactions) to control the photochromism of diarylethene (DAE). However, in these systems, the use of host-guest binding is still necessary to disrupt the stacking of hydrogen bonds and π-π interactions, in order to reproduce the photo-switching of the DAE structure (e.g., Mao, Y.; Liu, K.; Lv, G.; Wen, Y.; Zhu, X.; Lan, H.; Yi, T., Cb[8] Gated Photochromism of a Diarylethene Derivative Containing Thiazole Orange Groups. Chem. Commun. 2015, 51, 6667-6670). Alternatively, an external host-guest binding is required to spatially lock the DAE structure by introducing steric hindrance in the host cavity (e.g., Wang, J.; Avram, L.; Diskin-Posner, Y.; Białek, M. J.; Stawski, W.; Feller, M.; Klajn, R., Altering the Properties of Spiropyran Switches Using Coordination Cages with Different Symmetries. J. Am. Chem. Soc. 2022, 144, 21244-21254). This renders the photo-switching ineffective under UV and visible light, making it challenging to achieve specificity in locking (or unlocking) the fluorescence switching of photoactive materials solely through a single molecule system containing the DAE structure.

### SUMMARY

In one aspect, the present invention is directed to a [1]rotaxane with an acid-base controllable nanobent lasso to manipulate a photo-switching unit in the [1]rotaxane. The [1]rotaxane comprising a plurality of moieties, which sequentially comprise a macrocyclic moiety, a photo-switching moiety with photochromic property, a first station moiety, a threaded moiety, a second station moiety, and a stopper moiety with an aggregation-induced emission group. The macrocyclic moiety comprises a cyclic polyether moiety. The photo-switching moiety is bonded to the macrocyclic moiety. The photo-switching moiety performs a ring-closing reaction upon ultraviolet light exposure and a ring-opening reaction upon visible light exposure under a basic condition, and a chemical structure of the photo-switching moiety is -B-A-B-, wherein A is

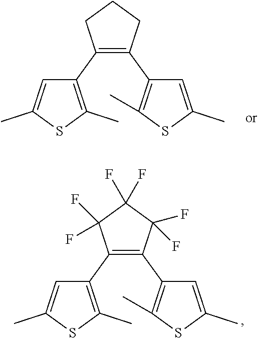

### B is,

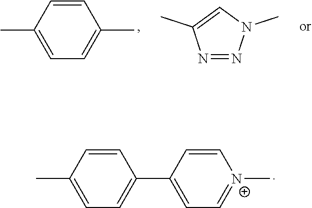

The first station moiety is bonded to the photochromic photo-switching moiety. The first station moiety is bonded to the photochromic photo-switching moiety, wherein the first station moiety comprises a secondary ammonium group to form a plurality of hydrogen bonding with the macrocyclic moiety when the first station moiety enters the macrocyclic moiety. The threaded moiety is bonded to the first station moiety and penetrating the macrocyclic moiety. The second station moiety is bonded to the threaded moiety, wherein the second station moiety forms a plurality of hydrogen bonding with the macrocyclic moiety when the second station moiety enters the macrocyclic moiety. The stopper moiety is bonded to the second station moiety, wherein the stopper comprises a tetraarylethylene moiety, and the photo-switching moiety and the stopper moiety are located on two sides of the macrocyclic moiety.

According to an embodiment, the macrocyclic moiety comprises a dibenzo-24-crown-8 moiety.

According to another embodiment, the secondary ammonium group comprises dialkylene ammonium moiety.

According to yet another embodiment, the threaded moiety comprises-(CH₂)ₙ—, and n=6-16.

According to yet another embodiment, the second station moiety is

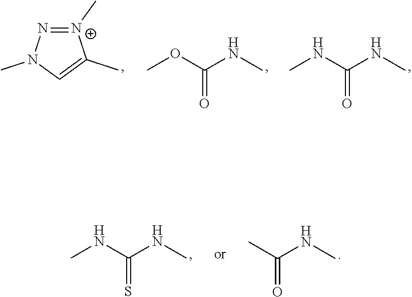

According to yet another embodiment, the tetraarylethylene moiety comprises substituted or unsubstituted tetraphenylethylene.

According to yet another embodiment, the [1]rotaxane further comprises a first linking moiety between the macrocyclic moiety and the photo-switching moiety, wherein the first linking moiety comprises —COO—(CH₂)ₙ—, n=1-3, and the —COO— moiety is bonded to the macrocyclic moiety.

According to yet another embodiment, the [1]rotaxane further comprises a second linking moiety between the photo-switching moiety and the first station moiety, wherein the second linking moiety comprises —(CH₂)ₙ—, and n=1-9.

According to yet another embodiment, the [1]rotaxane further comprises a third linking moiety between the second station moiety and the stopper moiety, wherein the third linking moiety comprises —(CH₂)ₙ—O—, and n=1-10, and the —O— moiety is bonded to the stopper moiety.

In another aspect, the present invention is also directed to a nanoparticle, which comprises the [1]rotaxane described above, and a micelle encompassing the [1]rotaxane.

According to an embodiment, the micelle comprises 2-[2-(2-hydroxyethoxy) propoxy]ethanol.

The foregoing presents a simplified summary of the disclosure to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

To make the disclosed technology and other objectives, features, advantages, and embodiments more clearly understood, the explanations of the accompanying drawings are provided as follows:

FIG. 1 illustrates a schematic cartoon structure depicting the unfolding of various moieties of a [1]rotaxane, which is a nanobent lasso controlled by acid-base to manipulate the optical switch of the [1]rotaxane, according to one embodiment of this disclosure.

FIG. 2 shows a schematic diagram of reversible transformations in three chemical structures, [1]RotA, [1]RotB-O, and [1]RotB-C, regulated by acid-base and light exposure at different wavelengths, according to one embodiment of this disclosure.

FIG. 3 shows a schematic diagram of methods for controlling the yields of isomers [1]RotA and NIM separately, according to one embodiment of this disclosure.

FIG. 4 presents a synthetic scheme for the chemical structure of [1]RPA, which is similar to the [1]RotA structure shown in FIG. 2, according to one embodiment of this disclosure.

FIG. 5A shows the relationship between three types of nanoparticles, NPsA, NPsB-O, and NPsB-C, containing the chemical structures [1]RotA, [1]RotB-O, and [1]RotB-C, respectively, and the generation of singlet oxygen, according to one embodiment of this disclosure.

FIG. 5B shows a light control image-guided photodynamic therapies (PDTs) in tumor tissues, according to one embodiment of this disclosure.

FIG. 6A demonstrates the use of nanoparticles NPsB in the logic circuit represented by the algebraic equation “((Input 1×Input 3)+((Input 2)×Input 3),” according to one embodiment of this disclosure.

FIG. 6B illustrates the truth value definition of the output of the logic circuit shown in FIG. 6A, according to one embodiment of this disclosure.

### DETAILED DESCRIPTION

To provide a more detailed explanation of various embodiments of the present disclosure, the following is accompanied by illustrations. It should be understood that when a “moiety” is referred to as being “bonded” to another moiety, it can indicate that this moiety is directly bonded to the other moiety, or it can also be bonded to the other moiety through an intermediate linking moiety. Conversely, when a moiety is referred to as being “directly bonded to another moiety,” it can be understood that, in this case, the absence of an intermediate linking moiety is explicitly defined.

### Chemical Structure of Acid-Base Controlled Nano-Bending Lassos to Manipulate Photo-Switching Units in [1]Rotaxanes

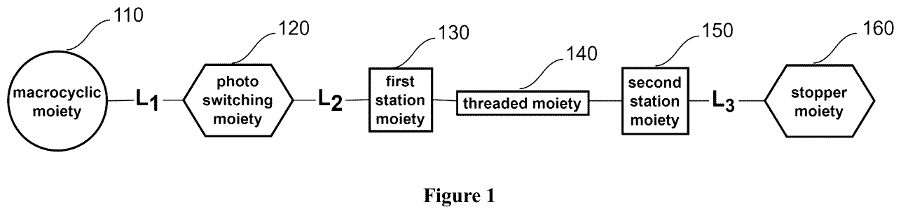

In the present disclosure, a single mechanically interlocked molecule (MIM) is used as the pure substance for a molecular switch. FIG. 1 illustrates a schematic cartoon structure depicting the unfolding of various moieties of a [1]rotaxane, which is a nanobent lasso controlled by acid-base to manipulate the optical switch of the [1]rotaxane, according to one embodiment of this disclosure.

In FIG. 1, the [1]rotaxane comprising a plurality of moieties, which sequentially comprise a macrocyclic moiety 110, a photo-switching moiety 120 with photochromic property, a first station moiety 130, a threaded moiety 140, a second station moiety 150, and a stopper moiety 160 with an aggregation-induced emission group.

The macrocyclic moiety 110 includes a cyclic polyether moiety. The macrocyclic moiety 110, for example, may contain a dibenzo-24-crown-8 (DB24C8) moiety.

The photo-switching moiety 120, responsible for photochromism, is bonded to the macrocyclic moiety 110. The photo-switching moiety 120 can undergo a ring-closing reaction or a ring-opening reaction upon exposure to light of different wavelengths. The chemical structure of the photo-switching moiety 120 is -B-A-B-, wherein A is

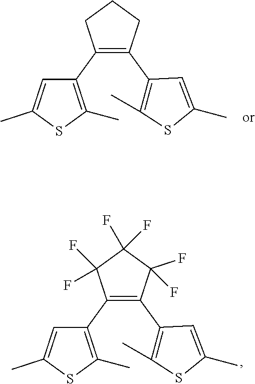

### B is,

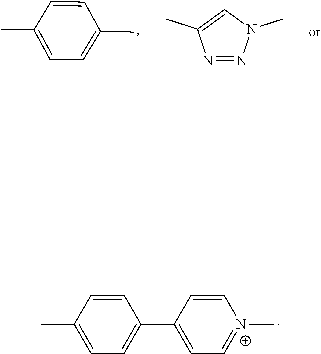

Therefore, The photo-switching moiety 120 performs a ring-closing reaction upon ultraviolet light exposure and a ring-opening reaction upon visible light exposure under a basic condition.

Between the macrocyclic moiety 110 and the photo-switching moiety 120, a first linking moiety L1 can optionally be inserted to create some distance between the macrocyclic moiety 110 and the photo-switching moiety 120. Since the function of the first linking moiety L1 is only to increase the distance between the macrocyclic moiety 110 and the photo-switching moiety 120, it is only necessary that the first linking moiety L1 does not undergo a chemical reaction in the environment of the [1]rotane. The first linking moiety L1 may be [macrocyclic moiety 110]—COO—(CH₂)ₙ—[photo-switching moiety 120], where n=1-3, for example.

The first station moiety 130 is bonded to the photo-switching moiety 120. When the first station moiety 130 enters the interior of the macrocyclic moiety 110, multiple hydrogen bonds are formed between the first station moiety 130 and the macrocyclic moiety 110. The first station moiety 130 contains a secondary ammonium group, such as a dialkylammonium moiety.

Optionally, a second linking moiety L2 may be inserted between the photo-switching moiety 120 and the first station moiety 130, creating some distance between the photo-switching moiety 120 and the first station moiety 130. Since the function of the second linking moiety L2 is solely to increase the distance between the photo-switching moiety 120 and the first station moiety 130, it is only necessary that the second linking moiety L2 does not undergo chemical reactions in the environment of this [1]rotaxane. The second linking moiety L2 may be [photo-switching moiety 120]—(CH₂)ₙ—[first station moiety 130], where n=1-9, for example.

The threaded moiety 140 is bonded to the first station moiety 130 and threads through the macrocyclic moiety 110 (this configuration is not shown in FIG. 1). The threaded moiety 140 may be —(CH₂)ₙ—, where n=6-16, for example.

The second station moiety 150 is bonded to the threaded moiety 140. When the second station moiety 150 enters the interior of the macrocyclic moiety 110, multiple hydrogen bonds are formed between the second station moiety 150 and the macrocyclic moiety 110. The chemical structure of the second station moiety 150 may be

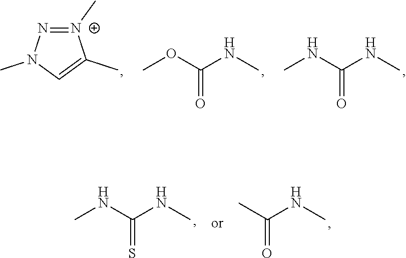

for example.

Due to the secondary ammonium group of the first station moiety 130 having a free proton, the protonation of the first station moiety 130 to have a positive charge can be controlled by adjusting the pH of the solution containing this [1]rotaxane. This determines whether the first station moiety 130 forms multiple hydrogen bonds with the ether groups of the macrocyclic moiety 110, anchoring the first station moiety 130 within the macrocyclic moiety 110. Specifically, when the first station moiety 130 is a secondary ammonium group, the first station moiety 130 carries a positive charge and can form multiple hydrogen bonds with the ether groups of the macrocyclic moiety 110, keeping the first station moiety 130 inside the macrocyclic moiety 110. Conversely, if the secondary ammonium group of the first station moiety 130 is deprotonated to become a neutral secondary amine group, the first station moiety 130 will exit the macrocyclic moiety 110. This allows the chemical structure of the second station moiety 150 to remain inside the macrocyclic moiety 110, forming multiple hydrogen bonds with the ether groups of the macrocyclic moiety 110.

The stopper moiety 160, containing an aggregation-induced emission (AIE) group, is bonded to the second station moiety 150. The stopper moiety 160 includes a tetraarylethylene (TAE) moiety, and the photo-switching moiety 120 and the stopper moiety 160 are located on opposite sides of the macrocyclic moiety 110. The tetraarylethylene moiety mentioned above includes substituted or unsubstituted tetraphenylethylene (TPE) moieties.

Optionally, a third linking moiety L3 may be inserted between the second station moiety 150 and the stopper moiety 160, allowing some distance between the second station moiety 150 and the stopper moiety 160. Since the function of the third linking moiety L3 is merely to increase the distance between the second station moiety 150 and the stopper moiety 160, it is only necessary that the third linking moiety L3 does not undergo chemical reactions in the environment of the [1]rotaxane. The third linking moiety L3 may, for example, be [second station moiety 150]—(CH₂)ₙ—O—[stopper moiety 160], where n=1-10.

In summary, one end of the threaded moiety 140 is connected to and threads through the macrocyclic moiety 110, and the other end of the threaded moiety 140 is attached to the stopper moiety 160. This allows the macrocyclic moiety 110 to move back and forth along the threaded moiety 140 without detaching, forming an intramolecular mechanically interlocked rotaxane configuration.

### Embodiment 1: Reversible Transformations of Chemical Structures of [1]RotA, [1]RotB-O, and [1]RotB-C

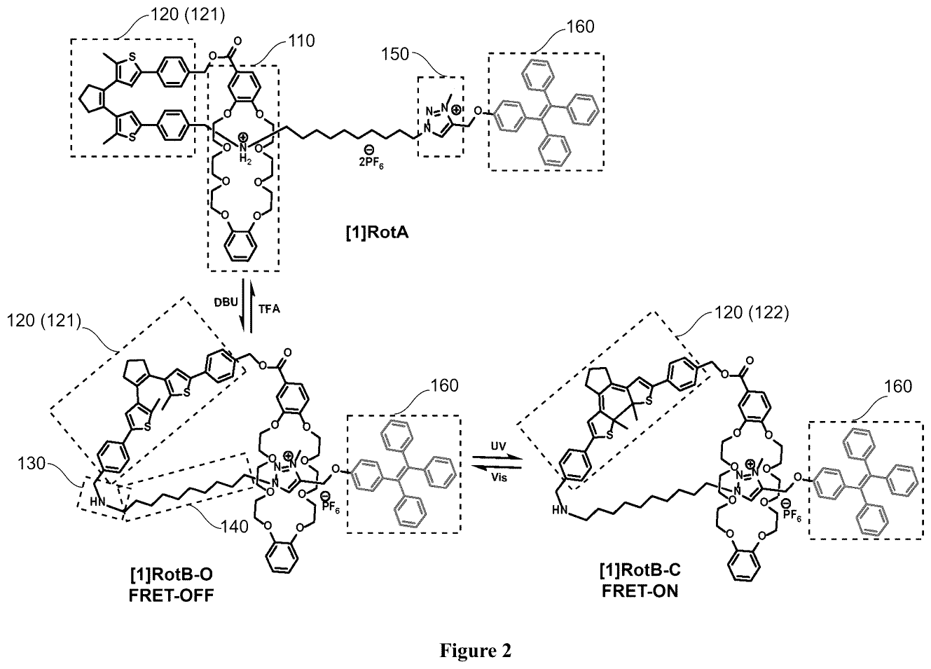

The following example illustrates the acid-base-controlled nanobent lasso to manipulate an optical switch in [1]rotaxane through the chemical structure transformations under three different environmental conditions. FIG. 2 shows a schematic diagram of reversible transformations in three chemical structures, [1]RotA, [1]RotB-O, and [1]RotB-C, regulated by acid-base and light exposure at different wavelengths, according to one embodiment of this disclosure.

In FIG. 2, the same or similar symbols are used to denote the various moieties corresponding to those in FIG. 1, with the addition of dashed boxes for clarity. The chemical structure of [1]RotA at the top of FIG. 2 is designed using a molecule with self-tightening properties and an asymmetrically threaded molecule strategy.

In the chemical structure of [1]RotA at the top, the macrocyclic moiety 110 is the dibenzo-24-crown-8 moiety, the photo-switching moiety 120 is

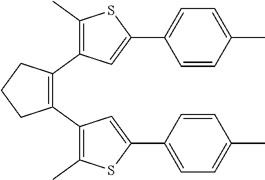 [SMILES: Cc1ccc(-c2cc(C3=C(c4cc(-c5ccc(C)cc5)sc4C)CCC3)c(C)s2)cc1]

with an ring opening structure 121, the first station moiety 130 is the dialkylammonium moiety (balanced with an anionic PF₆⁻ charge), the threaded moiety 140 is —(CH₂)₁₀—, the second station moiety 150 is

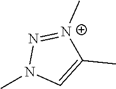

(N-methyltriazolium, abbreviated as MTA) (balanced with an anionic PF₆⁻ charge), and the stopper moiety 160 is tetraphenylethylene moiety with an aggregation-induced emission group. The first linking moiety L1 is [macrocyclic moiety 110]—COO—CH₂—[photo-switching moiety 120], the second linking moiety L2 is [photo-switching moiety 120]—CH₂—[first station moiety 130], and the third linking moiety L3 is [second station moiety 150]—CH₂—O—[stopper moiety 160].

In the chemical structure of [1]RotA, the dialkylammonium moiety of the first station moiety 130 carries a positive charge due to a free proton. Consequently, the dialkylammonium moiety of the first station moiety 130 resides within the macrocyclic moiety 110, allowing the free proton of the dialkylammonium moiety to form stable multiple hydrogen bonds with the multiple ether groups of the macrocyclic moiety 110, anchoring the first station moiety 130 within the macrocyclic moiety 110.

Upon addition of base (1,8-diazabicyclo[5.4.0]undec-7-ene, DBU), the dialkylammonium moiety of the first station moiety 130 in the chemical structure of [1]RotA undergoes deprotonation, resulting in the loss of its positive charge. At this point, the second station moiety 150, which retains a positive charge, is more favorable to stay within the macrocyclic moiety 110, relying on the multiple ether groups of the macrocyclic moiety 110 to stabilize the positive charge of the second station moiety 150

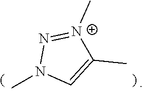

Therefore, after the addition of base, the chemical structure of [1]RotA transforms into the chemical structure of [1]RotB-O, where “O” represents the ring opening.

Conversely, if in the chemical structure of [1]RotB-O, the addition of acid (trifluoroacetic acid, TFA) would lead to the transformation of the chemical structure back into [1]RotA. Therefore, in [1]RotA, “A” represents acid, and in [1]RotB-C, “B” represents base.

Subsequently, upon irradiation with ultraviolet light (365 nm, 60 seconds), the ring opening structure 121 of the photo-switching moiety 120 in the chemical structure of [1]RotB-O undergoes a ring closing reaction, transforming the ring opening structure 121 into the ring closing structure122 of the photo-switching moiety 120 in the chemical structure of [1]RotB-C. In [1]RotB-C, “C” represents the ring closing. Conversely, the photo-switching moiety 120 in [1]RotB-C, when exposed to visible light (>500 nm, 600 seconds), can form the ring opening structure 121 of [1]RotB-O. Therefore, these two processes constitute a reversible reaction.

In the chemical structure of [1]RotB-C, the tetraphenylethylene (TPE) structure of the stopper moiety 160 serves as the donor in Förster Resonance Energy Transfer (FRET), while the ring closing structure 122 of the photo-switching moiety 120 acts as the acceptor in FRET. The ring closing structure 122 of the photo-switching moiety 120 absorbed the light emitted by the TPE structure of the stopper moiety 160 to complet the resonant energy transfer and quench the fluorescence of TPE structure of the stopper moiety 160. However, in the chemical structures of [1]RotA and [1]RotB-O, the ring opening structure 121 of the photo-switching moiety 120 is not the acceptor in FRET. Therefore, after absorbing ultraviolet light at 310 nm by the TPE structure of the stopper moiety 160, [1]RotA and [1]RotB-O lack an acceptor in resonant energy transfer, resulting in the emission of blue light at 470 nm from the TPE structure of the stopper moiety 160. The fluorescence excitation and emission wavelengths for the various structures are summarized in the table below (Table 1).

TABLE 1
Fluorescence Excitation and Emission Wavelengths of [1]RotA,
[1]RotB-O, and [1]RotB-C.

| | Fluorescence Excitation | Fluorescence Emission |
|---|---|---|
| Structure | Wavelength | Wavelength |
| [1]RotA | 310 nm (UV) | 470 nm (Blue light) |
| [1]RotB-O | 310 nm (UV) | 470 nm (Blue light) |
| [1]RotB-C | 310 nm (UV) | Quenched |

### Embodiment 2: Method for Controlling Synthesis of Isomers [1]RotA or NIM Chemical Structures

Comparing the chemical structures of [1]RotA and NIM, which is an isomer of [1]RotA, the difference lies in the threaded moiety 140 between the first station moiety 130 and the second station moiety 150 in the chemical structure of [1]RotA and NIM. The threaded moiety 140 threads through the macrocyclic moiety 110 in [1]RotA chemical structure, while the threaded moiety 140 is located outside the macrocyclic moiety 110 in NIM chemical structure.

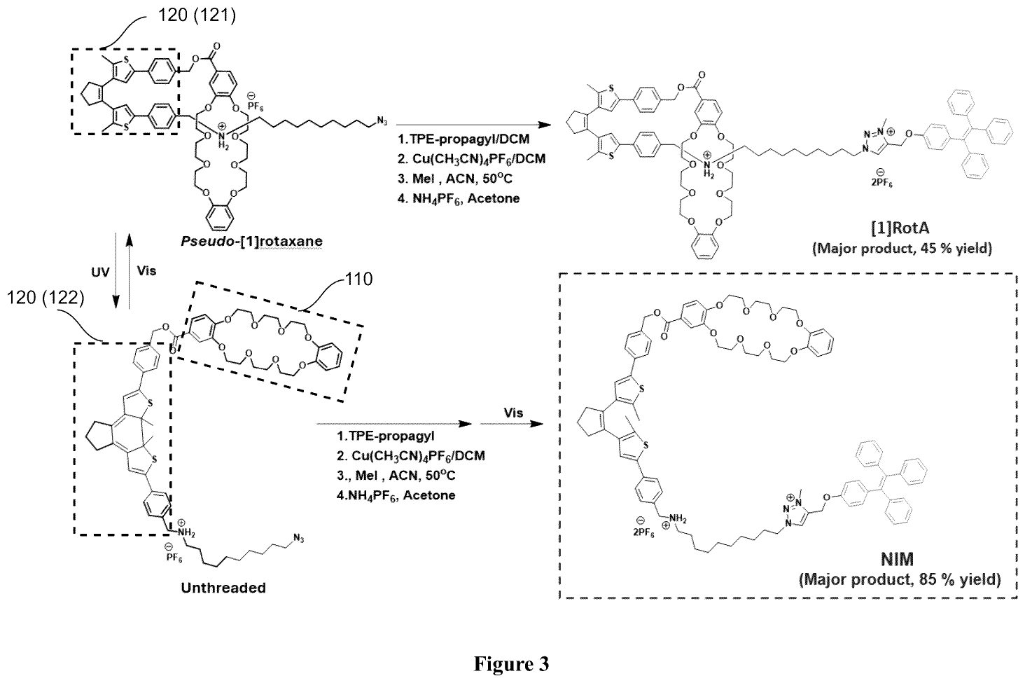

FIG. 3 shows a schematic diagram of methods for controlling the yields of isomers [1]RotA and NIM separately, according to one embodiment of this disclosure. Please refer to both FIG. 2 and FIG. 3. In the upper part of FIG. 3, when the photo-switching moiety 120 is in the ring opening structure 121, the structure of the photo-switching moiety 120 is more flexible and bendable, allowing the threaded moiety 140 to thread through the macrocyclic moiety 110. In the lower part of FIG. 3, when the photo-switching moiety 120 is in the ring closing structure 122, the structure of the photo-switching moiety 120 is rigid and cannot bend, preventing the threaded moiety 140 from threading through the macrocyclic moiety 110 to be located outside the macrocyclic moiety 110. As the ring opening structure 121 and ring closing structure 122 of the photo-switching moiety 120 can be transformed by irradiating with ultraviolet or visible light, the structures of the photo-switching moiety 120 can be controlled by illuminating with different wavelengths of light, thus determining whether to synthesize the chemical structure of [1]RotA or NIM.

In the upper part of FIG. 3, it was observed that when a pseudo-[1]rotaxane was used as the starting material, a series of reactions involving the addition of the second station moiety 150 and stopper moiety 160 to the end of the threaded moiety 140 resulted in the main product of [1]rotaxane (i.e., [1]RotA) with a yield of 45%. In the lower part of FIG. 3, it was seen that when using the “unthreaded structure” as the starting material, a series of reactions involving the addition of the second station moiety 150 and stopper moiety 160 to the end of the threaded moiety 140, followed by irradiation with visible light to restore the photo-switching moiety 120 to the ring closing structure 121, led to the main product of the NIM structure with a yield of 85%.

### Embodiment 3: Synthesis of [1]Rotaxane and [1]RPA Chemical Structures

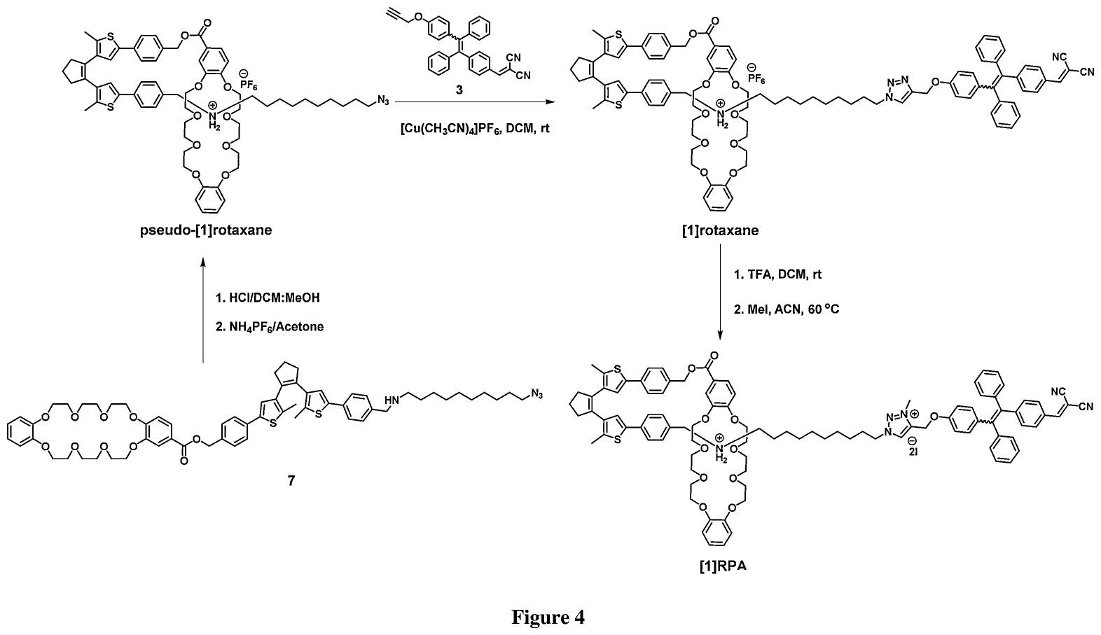

FIG. 4 presents a synthetic scheme for the chemical structure of [1]RPA, which is similar to the [1]RotA structure shown in FIG. 2, according to one embodiment of this disclosure. The key difference between the chemical structures of [1]RotA and [1]RPA lies in the structure of the tetraphenylethylene moiety in the stopper moiety 160. In the chemical structure of [1]RotA, the tetraphenylethylene moiety is unsubstituted, whereas in the chemical structure of [1]RPA, the tetraphenylethylene moiety has a substituent of

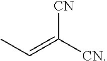

While FIG. 4 illustrates the synthetic pathway using the [1]RPA chemical structure as an example, a similar synthetic pathway can be employed for the synthesis of the [1]RotA chemical structure. In the second stage of the reactions depicted in FIG. 4, compound 3 with the unsubstituted

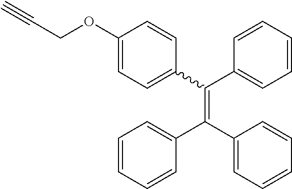 [SMILES: C#CCOc1ccc(C(=C(c2ccccc2)c2ccccc2)c2ccccc2)cc1]

can be used instead of

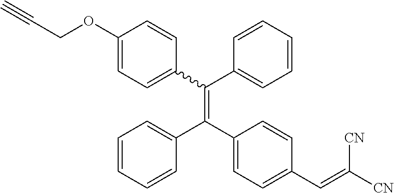 [SMILES: C#CCOc1ccc(C(=C(c2ccccc2)c2ccc(C=C(C#N)C#N)cc2)c2ccccc2)cc1]

to synthesize the [1]RotA chemical structure.

In the first stage of the reaction depicted in FIG. 4, compound 7 (2.25 g, 1.99 mmol) was dissolved in a 1:1 mixture of dichloromethane (DCM) and methanol (MeOH), and 2 mL of hydrochloric acid was added. The mixture was stirred overnight at room temperature. The resulting mixture was extracted with DCM, followed by washing with 50 mL of deionized water twice. After solvent removal, the chloride salt of compound 7 was obtained. Subsequently, the compound was evenly dispersed in 50 mL of acetone, and 10 mL of a saturated solution of ammonium hexafluorophosphate (NH₄PF₆) was added. The mixture was stirred overnight at room temperature. After solvent removal, the residual material was washed with 50 mL of deionized water three times. Finally, the washed solid compound was dried under vacuum for 24 hours, yielding a pale purple solid of pseudo-[1]rotaxane. This compound could be directly used for the synthesis of the intermediate [1]rotaxane.

In the second stage of the reaction illustrated in FIG. 4, pseudo-[1]rotaxane (1 g, 0.7 mmol) was dissolved in 50 mL of dry and degassed dichloromethane. The mixture was stirred continuously at room temperature for 24 hours. Subsequently, compound 3 (389 mg, 0.84 mmol) and copper (I) tetrafluoroborate acetonitrile complex ([Cu(CH₃CN)₄]PF₆) (313 mg, 0.84 mmol) were added to the reaction, and the stirring was continued for an additional 24 hours in a nitrogen atmosphere at room temperature. The reaction solution was then diluted with 100 mL of dichloromethane and washed with a solution of ethylenediaminetetraacetic acid disodium salt (Na₂EDTA) (0.1 M, 2×100 mL) and water (2×50 mL). After drying over anhydrous sodium sulfate (Na₂SO₄) and solvent removal under reduced pressure, the residue was subjected to column chromatography (DCM:MeOH=100:1) to obtain an orange solid of the intermediate [1]rotaxane with a yield of 40.26% (474 mg).

In the third stage of the reaction depicted in FIG. 4, the intermediate [1]rotaxane (400 mg, 0.25 mmol), dissolved in 10 mL of acetonitrile (ACN), was combined with 5 mL of methyl iodide (Mel) and stirred overnight at 60° C. in an oil bath. After cooling to room temperature, the solvent was removed under reduced pressure. The resulting residue was washed three times with an excess of Mel using 50 mL of diethyl ether for each wash. Subsequently, two extractions were performed using 100 mL of dichloromethane for each. The residue was then washed twice with 40 mL of deionized water, dried over anhydrous sodium sulfate, and concentrated under reduced pressure. The remaining material was purified by flash column chromatography (DCM:MeOH=50:1) to yield an orange solid of [1]RPA with a yield of 85% (395 mg).

### Embodiment 4: Controlling the Generation of Singlet Oxygen Molecules Using Nanoparticles NPsA, NPsB-O, or NPsB-C Containing [1]RPA, [1]RPB-O, or [1]RPB-C Chemical Structures

Due to the low physiological toxicity of the [1]RPA molecules mentioned above to the human body and their propensity to generate singlet oxygen molecules, aggregation-induced emission (AIE) can be induced by the

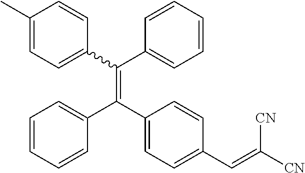 [SMILES: Cc1ccc(C(=C(c2ccccc2)c2ccc(C=C(C#N)C#N)cc2)c2ccccc2)cc1]

moiety of [1]RPA. This property makes [1]RPA suitable for use in photodynamic therapy. The

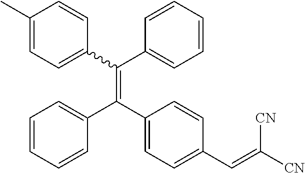 [SMILES: Cc1ccc(C(=C(c2ccccc2)c2ccc(C=C(C#N)C#N)cc2)c2ccccc2)cc1]

moiety is employed as a photosensitizer, utilizing its ability to generate singlet oxygen molecules for photodynamic therapy. In photodynamic therapy, the singlet oxygen molecules produced are used to induce phototoxicity in specific cancer cells or diseased cells, achieving therapeutic effects.

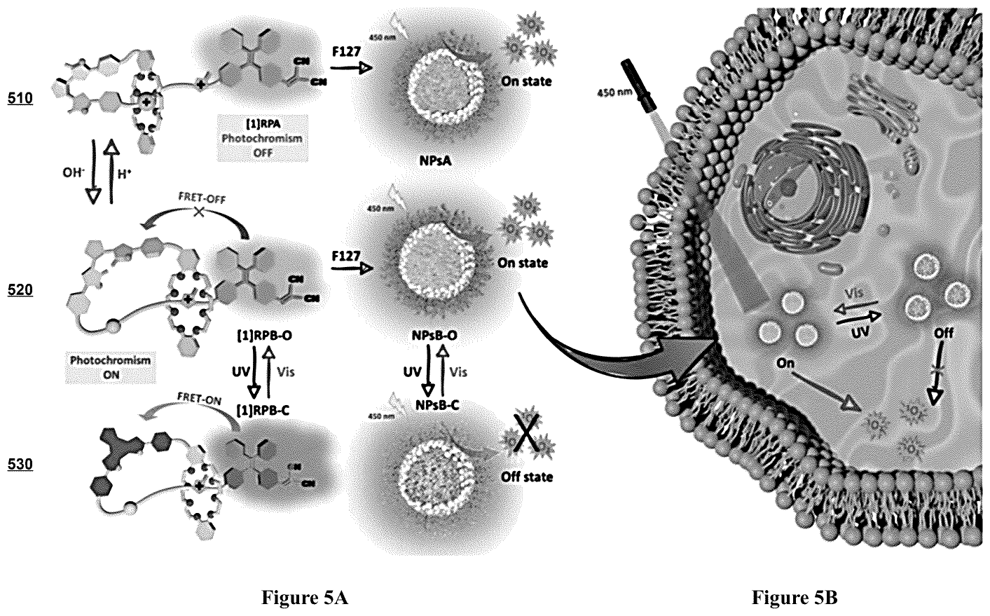

FIG. 5A shows the relationship between three types of nanoparticles, NPsA, NPsB-O, and NPsB-C, containing the chemical structures [1]RotA, [1]RotB-O, and [1]RotB-C, respectively, and the generation of singlet oxygen, according to one embodiment of this disclosure.

In the method 510 of FIG. 5A, [1]RPA was mixed with the surfactant 2-[2-(2-hydroxyethoxy) ethoxy]ethanol (commercially known as Pluronic F-127, a non-ionic surfactant). Pluronic F-127, when mixed with [1]RPA, forms a nanostructure of micelles in the aqueous solution, encapsulating [1]RPA and creating suspended nanoparticles NPsA in the aqueous solution. In the chemical structure of [1]RPA, the dialkylammonium moiety of the first station part 130 remained within the dibenzo-24-crown-8 (DB24C8) moiety of the macrocyclic moiety 110, maintaining a contracted conformation crucial for synthesizing the [1]RPA structure shown in FIG. 4. Within the [1]RPA chemical structure, only the

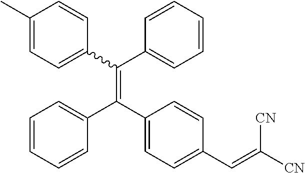 [SMILES: Cc1ccc(C(=C(c2ccccc2)c2ccc(C=C(C#N)C#N)cc2)c2ccccc2)cc1]

of the stopper moiety 160 could absorb ultraviolet light (450 nm) and emit red light at 595 nm, allowing the absorption of 595 nm red light by triplet oxygen molecules to convert the triplet oxygen molecules into highly reactive singlet oxygen molecules.

In method 520 of FIG. 5A, a base (OH—) can be added to the solution of the [1]RPA chemical structure to transform the [1]RPA chemical structure into the [1]RPB-O chemical structure. Conversely, an acid can be added to the solution of the [1]RPB-O chemical structure to reversely convert the [1]RPB-O chemical structure back to the [1]RPA chemical structure. Subsequently, mixing the [1]RPB-O chemical structure with the surfactant Pluronic F-127 in the aqueous solution forms micelles encapsulating the [1]RPB-O chemical structure, resulting in nano-sized particles NPsB-O. Similarly, within the [1]RPB-O chemical structure,

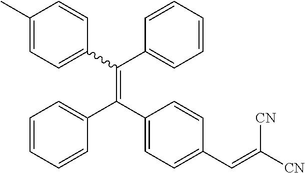 [SMILES: Cc1ccc(C(=C(c2ccccc2)c2ccc(C=C(C#N)C#N)cc2)c2ccccc2)cc1]

only the of the stopper moiety 160 serves as a photosensitizer, absorbing ultraviolet light (450 nm) and emitting red light at 595 nm. This allows triplet oxygen molecules to absorb 595 nm red light to convert the triplet oxygen molecules into highly reactive singlet oxygen molecules.

Regarding the detailed transformation of [1]RPA into the [1]RPB-O chemical structure, in method 520 of FIG. 5A, the addition of the base to the [1]RPA chemical structure neutralizes the free protons of the dimethylammonium moiety in the first station moiety 130, causing the dimethylammonium moiety to lose its positive charge and move out of the dibenzo-24-crown-8 (DB24C8) moiety of the macrocyclic moiety 110. Consequently, the threaded moiety 140 is driven to move within the dibenzo-24-crown-8 (DB24C8) moiety, relocating the

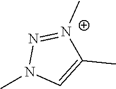

moiety of the second station moiety 150 inside the dibenzo-24-crown-8 (DB24C8) moiety, forming a more relaxed conformation. This shortens the distance between the dithienylethene (DTE) moiety of the photo-switching moiety 120 and the

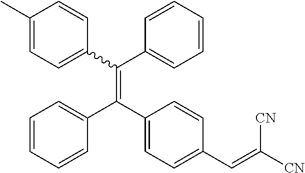 [SMILES: Cc1ccc(C(=C(c2ccccc2)c2ccc(C=C(C#N)C#N)cc2)c2ccccc2)cc1]

moiety of the stopper moiety 160.

In method 530 of FIG. 5A, the [1]RPB-O chemical structure can be subjected to 365 nm ultraviolet light for 60 seconds, inducing a ring-closing reaction of the dithienylethene (DTE) moiety in the photo-switching moiety 120 to obtain the [1]RPB-C chemical structure. Conversely, the [1]RPB-C chemical structure can be exposed to visible light (>500 nm) for 600 seconds, causing a ring-opening reaction of the DTE moiety in the photo-switching moiety 120 to reverse the chemical structure back to [1]RPB-O. The fluorescence excitation and emission wavelengths for [1]RPA, [1]RPB-O, and [1]RPB-C are listed in Table 2 below.

TABLE 2
Fluorescence Excitation and Emission Wavelengths of
[1]RPA, [1]RPB-O, and [1]RPB-C.

| | Fluorescence Excitation | Fluorescence Emission |
|---|---|---|
| Structure | Wavelength | Wavelength |
| [1]RPA | 450 nm (Blue light) | 595 nm (Red light) |
| [1]RPB-O | 450 nm (Blue light) | 595 nm (Red light) |
| [1]RPB-C | 450 nm (Blue light) | Quenched |

Subsequently, the [1]RPB-C chemical structure was mixed with the surfactant Pluronic F-127 in an aqueous solution to form micelles embedding the [1]RPB-C chemical structure, resulting in nanoparticles NPsB-C. Due to the presence of the

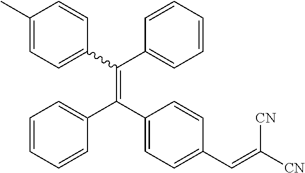 [SMILES: Cc1ccc(C(=C(c2ccccc2)c2ccc(C=C(C#N)C#N)cc2)c2ccccc2)cc1]

moiety in the stopper part 160 of the [1]RPB-C chemical structure, which could absorb ultraviolet light at 450 nm and emit red light at 595 nm, and the close proximity of the ring closing structure

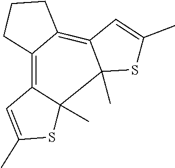

in the photo-switching moiety 120, which competed with singlet oxygen for the absorption of 595 nm red light. Therefore, after exposure to ultraviolet light, the nanoparticles NPsB-C could not facilitate the conversion of triplet oxygen into highly reactive singlet oxygen.

As explained in the context of FIG. 5A, when the photo-switching moiety 120 of this [1]rotaxane has an ring opening structure 121, i.e., when using nanoparticles NPsA or NPsB-O containing [1]RPA or [1]RPB-O, triplet oxygen molecules can be excited to singlet oxygen by irradiating with ultraviolet light. However, when the photo-switching moiety 120 of the [1]rotaxane has a ring closing structure 122, i.e., when using nanoparticles NPsB-C containing [1]RPB-C, singlet oxygen cannot be generated.

FIG. 5B shows a light control image-guided photodynamic therapies (PDTs) in tumor tissues, according to one embodiment of this disclosure. By delivering nanoparticles NPsB-O into tumor cells and then controlling the content of nanoparticles NPsB-O and NPsB-C in tumor cells using 365 nm ultraviolet light and 450 nm blue light, the generation amount of singlet oxygen molecules in tumor cells can be controlled. The higher the content of nanoparticles NPsB-O, the more singlet oxygen molecules can be generated upon irradiation with 450 nm blue light, causing damage and subsequent death of tumor cells in the tumor tissue.

As mentioned above, the chemical structures of [1]RPA, [1]RPB-O, and [1]RPB-C serve as multi-stimuli photosensitizers that can be controlled through a dual-switch mechanism involving pH value adjustment and UV-Vis light. The generation of toxic singlet oxygen molecules is manipulated. In the acidic environment of cancer cells, [1]rotaxane undergoes a contracted lasso conformation (where the first station moiety 130 remains within the macrocyclic moiety 110), preventing the formation of a ring closing structure in the photo-switching moiety 120 under ultraviolet light. Consequently, the Förster Resonance Energy Transfer (FRET) energy transfer cannot occur. Thus, [1]rotaxane can utilize the light emitted by the photosensitizer to excite oxygen and convert oxygen into toxic singlet oxygen molecules, facilitating photodynamic therapy (PDT) for killing tumor cells.

Conversely, in the neutral or alkaline environment of normal cells, [1]rotaxane adopts an extended lasso conformation (where the second station moiety 150 remains within the macrocyclic moiety 110). During this condition, the ring closing reaction can occur in the photo-switching moiety 120 under ultraviolet light, allowing FRET energy transfer to take place. This prevents [1]rotaxane from utilizing the light emitted by the photosensitizer to excite oxygen and generate toxic singlet oxygen molecules. Therefore, [1]rotaxane, when exposed to ultraviolet light, cannot induce cell death in tumor cells.

The concept of photodynamic therapy using molecular dual-switches (i.e., [1]rotaxane) in these nanoparticles effectively provides controlled production and management of toxic singlet oxygen (¹O₂) output. This system serves as a promising approach for targeted therapy and applications in future photodynamic treatments. The multi-stimulus responsive supramolecular [1]rotaxane system has potential applications in the biomedical and clinical medical industries.

### Embodiment 5: Logic Circuit of Nanoparticles NPsB

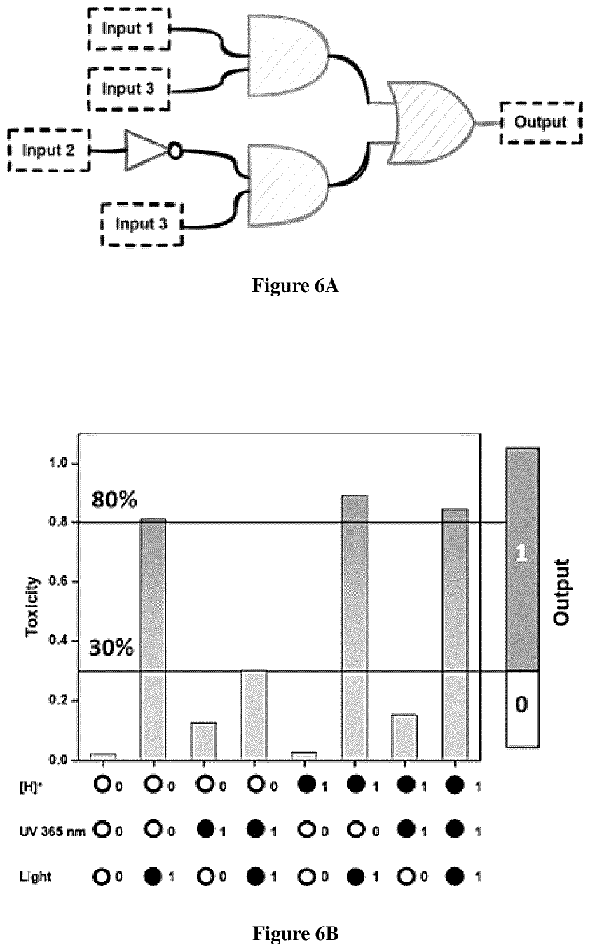

In this embodiment, utilizing the dual-state nature of nanoparticles NPsB (NPsB-O and NPsB-C), a logic circuit is constructed using these nanoparticles to provide controlled production and management of toxic singlet oxygen (102) output. FIG. 6A demonstrates the use of nanoparticles NPsB in the logic circuit represented by the algebraic equation “((Input 1×Input 3)+((Input 2)×Input 3),” according to one embodiment of this disclosure. FIG. 6B shows the output of the logic circuit depicted in FIG. 6A, representing the selectively toxic pathway to eliminate Hela cells. The truth table for the output of the logic circuit in FIG. 6A is presented in the table below (Table 3).

TABLE 3
Truth table

| Input1 | Input2 | Input3 | | |
|---|---|---|---|---|
| [H⁺] | [365 nm UV] | [450 nm Blue light] | Output | Toxicity |
| 0 | 0 | 0 | 0 | low (<30%) |
| 0 | 0 | 1 | 1 | high (>80%) |
| 0 | 1 | 0 | 0 | low (<30%) |
| 0 | 1 | 1 | 0 | low (<30%) |
| 1 | 0 | 0 | 0 | low (<30%) |
| 1 | 0 | 1 | 1 | high (>80%) |
| 1 | 1 | 0 | 0 | low (<30%) |
| 1 | 1 | 1 | 1 | high (>80%) |

In Table 3, within this [1]rotaxane system, the input of [H⁺] (Input 1) can control whether the first station moiety 130 or the second station moiety 150 will stay within the macrocyclic moiety 110. The Input 2 [365 nm UV light] can control the structure of the photo-switching moiety 120 to be either a ring opening structure (O) or a ring closing structure (C). The Input 3 [450 nm blue light] can control the generation of singlet oxygen molecules. Therefore, using the truth table in Table 3, one can determine the required amount of singlet oxygen molecules based on the needs of tumor therapy and correspondingly find the combination conditions of Inputs 1-3.

In summary, through the mechanical interlocking structure in NPsB and the reversible contraction/expansion of [1]rotaxane, a sequential pH-controlled locking/unlocking photoswitch can be achieved in a reversible FRET process. This system can be used to regulate the generation of singlet oxygen and can be programmed to output toxic pathways in a molecular machine.

As evident from the above-described embodiments of the present invention, the multifunctionality of the [1]rotaxane system with a dual-functional and sequentially locked/unlocked photoswitch containing a diphenylethene (DAE) structure allows the manipulation of different effects of resonance energy transfer through the contraction/expansion of the [1]rotaxane nano lasso. Furthermore, under the influence of the tightened lasso state due to acid regulation, the original photo-switchable diphenylethene (DAE) structure is locked in the closed-ring form as a result of the tightened lasso. If the nano-loops are relaxed to form a loosened lasso through an alkaline reaction, the diphenylethene (DAE) structure, released due to the loosened lasso, will restore the photo-switchable state. Thus, UV and visible light irradiation can induce reversible reactions for closed-ring and open-ring photo transformations, controlling Forster Resonance Energy Transfer (FRET) and fluorescence color changes. The contraction (tightened lasso) and relaxation (loosened lasso) of the nano lasso, regulated by acidic and alkaline conditions, allow non-invasive control of singlet oxygen molecule (¹O₂) production. This system can be used in the construction of logic circuits. These [1]rotaxanes possess the capability of responding to multiple stimuli, making them suitable for creating smart materials on a nanoscale platform. The [1]rotaxanes can be used for selective labeling of cancer cells and targeted therapy through photodynamic treatment, showing significant potential for selective photodynamic clinical therapy in the future.

In summary, the design of the [1]rotaxane structure with acid-base control of the nano lasso allows manipulation of the photo-switching behavior. Under acidic conditions, the contracted nano-latch loop exists in a tightened state, resulting in the chemical structure [1]RotA or [1]RPA. In this state, the contracted nano-latch loop inhibits the reversible ring opening and closing function of the photo-switching moiety 120 (i.e., DAE or DTE) when exposed to UV light (365 nm).

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. A [1]rotaxane with an acid-base controllable nanobent lasso to manipulate a photo-switching unit in the [1]rotaxane, the [1]rotaxane comprising a plurality of moieties, which sequentially comprise:
a macrocyclic moiety comprising a cyclic polyether moiety;
a photo-switching moiety with photochromic property bonded to the macrocyclic moiety, wherein the photo-switching moiety performs a ring-closing reaction upon ultraviolet light exposure and a ring-opening reaction upon visible light exposure under a basic condition, and a chemical structure of the photo-switching moiety is -B-A-B-, wherein A is 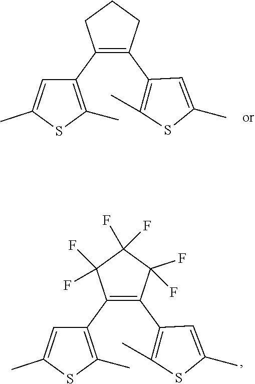
B is, 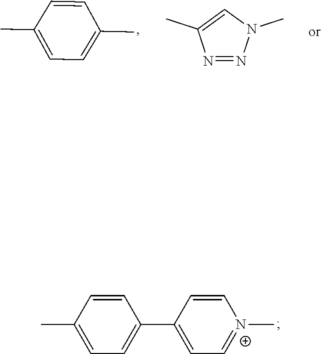
a first station moiety bonded to the photochromic photo-switching moiety, wherein the first station moiety comprises a secondary ammonium group to form a plurality of hydrogen bonding with the macrocyclic moiety when the first station moiety enters the macrocyclic moiety;
a threaded moiety bonded to the first station moiety and penetrating the macrocyclic moiety;
a second station moiety bonded to the threaded moiety, wherein the second station moiety forms a plurality of hydrogen bonding with the macrocyclic moiety when the second station moiety enters the macrocyclic moiety; and
a stopper moiety with an aggregation-induced emission group bonded to the second station moiety, wherein the stopper comprises a tetraarylethylene moiety, and the photo-switching moiety and the stopper moiety are located on two sides of the macrocyclic moiety.

2. The [1]rotaxane of claim 1, wherein the macrocyclic moiety comprises a dibenzo-24-crown-8 moiety.

3. The [1]rotaxane of claim 1, wherein the secondary ammonium group comprises dialkylene ammonium moiety.

4. The [1]rotaxane of claim 1, wherein the threaded moiety comprises —(CH₂)ₙ—, and n=6-16.

5. The [1]rotaxane of claim 1, wherein the second station moiety is

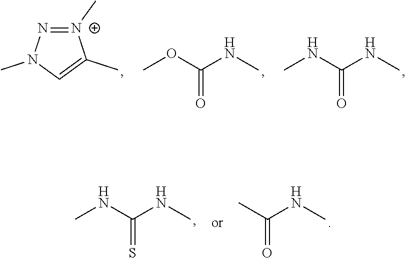

6. The [1]rotaxane of claim 1, wherein the tetraarylethylene moiety comprises substituted or unsubstituted tetraphenylethylene.

7. The [1]rotaxane of claim 1, further comprising a first linking moiety between the macrocyclic moiety and the photo-switching moiety, wherein the first linking moiety comprises —COO—(CH₂)ₙ—, n=1-3, and the —COO— moiety is bonded to the macrocyclic moiety.

8. The [1]rotaxane of claim 1, further comprising a second linking moiety between the photo-switching moiety and the first station moiety, wherein the second linking moiety comprises —(CH₂)ₙ—, and n=1-9.

9. The [1]rotaxane of claim 1, further comprising a third linking moiety between the second station moiety and the stopper moiety, wherein the third linking moiety comprises —(CH₂)ₙ—O—, and n=1-10, and the —O— moiety is bonded to the stopper moiety.

10. A nanoparticle, comprising:
the [1]rotaxane of claim 1; and
a micelle encompassing the [1]rotaxane.

11. The nanoparticles of claim 10, wherein the macrocyclic moiety comprises a dibenzo-24-crown-8 moiety.

12. The nanoparticles of claim 10, wherein the secondary ammonium group comprises dialkylene ammonium moiety.

13. The nanoparticles of claim 10, wherein the threaded moiety comprises —(CH₂)ₙ—, and n=6-16.

14. The nanoparticles of claim 10, wherein the second station moiety is

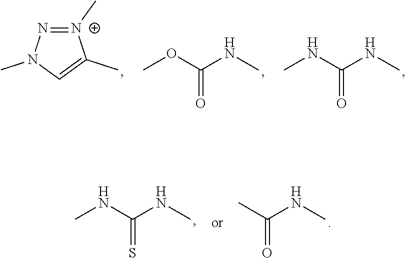

15. The nanoparticles of claim 10, wherein the tetraarylethylene moiety comprises substituted or unsubstituted tetraphenylethylene.

16. The nanoparticles of claim 10, further comprising a first linking moiety between the macrocyclic moiety and the photo-switching moiety, wherein the first linking moiety comprises —COO—(CH₂)ₙ—, n=1-3, and the —COO— moiety is bonded to the macrocyclic moiety.

17. The nanoparticles of claim 10, further comprising a second linking moiety between the photo-switching moiety and the first station moiety, wherein the second linking moiety comprises —(CH₂)ₙ—, and n=1-9.

18. The nanoparticles of claim 10, further comprising a third linking moiety between the second station moiety and the stopper moiety, wherein the third linking moiety comprises —(CH₂)ₙ—O—, and n=1-10, and the —O— moiety is bonded to the stopper moiety.

19. The nanoparticles of claim 10, wherein the micelle comprises 2-[2-(2-hydroxyethoxy)propoxy]ethanol.
